(19) United States
(12) Patent Application Publication
(10) Pub. No.: US 2019/0358201 A1
(43) Pub. Date: Nov. 28, 2019

(54) MODIFIED DOXORUBICIN COMPOSITIONS AND METHODS

(71) Applicant: NantCell, Inc., Culver City, CA (US)

(72) Inventors: John H. Lee, Culver City, CA (US); Patrick Soon-Shiong, Culver City, CA (US)

(21) Appl. No.: 16/418,793

(22) Filed: May 21, 2019

Related U.S. Application Data
(60) Provisional application No. 62/676,654, filed on May 25, 2018.
(60) Provisional application No. 62/679,283, filed on Jun. 1, 2018.

(51) Int. Cl. A61K 31/4025; A61P 35/00

(52) U.S. Cl. CPC A61K 31/4025; A61P 35/00

(57) ABSTRACT

The inventors unexpectedly discovered that aldoxorubicin lacks cardiotoxicity in patients treated with aldoxorubicin alone or in combination with ifosfamide/mesna. Notably, no evidence of cardiac toxicity of aldoxorubicin was found, even at doxorubicin equivalent cumulative doses of up to 10,000 mg/m².

This application claims priority to our U.S. provisional patent applications with the Ser. Nos. 62/676,654, filed May 25, 2018, and 62/679,283, filed Jun. 1, 2018, both of which are incorporated by reference in its entirety herein.

### FIELD OF THE INVENTION

The field of the invention is cancer treatment with pharmaceutical compositions that have significantly reduced toxicity, especially as it relates to cardiotoxicity using aldoxorubicin.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Doxorubicin is a compound known for many decades and used as DNA intercalator, and as such acts as topoisomerase inhibitor. Doxorubicin is employed as a chemotherapeutic drug for various cancers, and especially ovarian cancer, various sarcomas, and multiple myeloma. While at least some therapeutic effect can be achieved with doxorubicin, significant side effects are known, including fatigue, fever, vomiting, diarrhea, neutropenia, thrombocytopenia, and anemia. More serious side effects include severe myelosuppression, hepatic impairment, and cardiotoxicity. Indeed, doxorubicin has a black box warning for cardiotoxicity in the form of congestive heart failure indicated by significant decreases in left ventricular ejection fraction (LVEF). In view of this significant toxicity, the allowable maximum cumulative lifetime dose of doxorubicin is 550 mg/m².

To reduce toxicity, various efforts have been undertaken. For example, doxorubicin can be PEGylated and encapsulated in liposomes (marketed under the tradename Doxil). However, such form of doxorubicin is typically associated with hand-foot syndrome where doxorubicin preferentially accumulates in the skin due to the PEG moieties. To avoid hand-foot syndrome, doxorubicin can be administered in liposomal form without PEGylation (marketed under the tradename Myocet). While the toxicology profile of liposomal doxorubicin is at least to some degree improved, liposomal packing may lead to a different distribution profile in a patient and other altered pharmacodynamic and pharmacokinetic properties.

More recently, doxorubicin was modified with a reactive linker molecule that selectively binds to albumin upon injection (known as aldoxorubicin). Thus, albumin will be used in vivo as a carrier for doxorubicin, which preferentially delivers the so bound doxorubicin to the tumor microenvironment. Advantageously, the linker in aldoxorubicin is an acid-sensitive linker that preferentially releases the doxorubicin in the acidic tumor microenvironment. However, in a Phase 3 clinical trial of treatment of soft tissue sarcoma, Aldoxorubicin performed better than investigator's choice for the entire study population, but narrowly missed statistical significance in progression-free survival, or PFS (p=0.12; HR=0.81, 95% CI 0.64-1.06), which was the trial's primary endpoint. See Chawla et al, Phase III study of aldoxorubicin vs investigators' choice as treatment for relapsed/refractory soft tissue sarcomas (J. Clin Oncol. 2017; 35(15_suppl):11000). Similar data were also reported elsewhere (JAMA Oncol. 2015; 1(9):1272-1280). Moreover, as doxorubicin is ultimately released from the albumin carrier, the same issues with cardiotoxicity can be expected. Thus, some reports recommended a recommended doxorubicin equivalent dose of 260 mg/m² (Clin Cancer Res 2007; 13(16)), which limits the therapeutic effects.

Therefore, despite the common use of doxorubicin in cancer treatment, actual or expected serious side effects, and particularly cardiotoxicity, remain a substantial issue. Thus, there is still a need for improved compositions and methods for doxorubicin.

### SUMMARY OF THE INVENTION

Despite release of doxorubicin from albumin in acidic tissue environment, the inventors unexpectedly discovered that aldoxorubicin improved antitumor activity, and exhibited alone, or in combination with ifosfamide, lack of cardiac toxicity. Most notably, while the allowable maximum cumulative lifetime dose of doxorubicin is 550 mg/m², no cardiotoxicity was observed in patients who received aldoxorubicin with doxorubicin equivalent doses even beyond 3000 mg/m². Furthermore, the inventors unexpectedly observed that doxorubicin after modification with N-c-maleimidocaproic acid hydrazide did not lead to significant quantities of doxorubicinol, which is the doxorubicin metabolite suspected to be a significant contributor to cardiotoxicity.

The inventive subject matter is directed to various methods of treating a disease in a patient by administering a modified doxorubicin. Thus, one aspect of the inventive subject matter includes a method of treating cancer in a patient, comprising: administering a carbonyl-modified doxorubicin to the patient at a cumulative dosage equivalent to doxorubicin of at least 4,000 mg/m², or at least 7,000 mg/m², or at least 10,000 mg/m²; and wherein the step of administering the carbonyl-modified doxorubicin does not produce cardiotoxicity. Preferably, the carbonyl-modified doxorubicin is contemplated to be aldoxorubicin. The method may also include a step of measuring doxorubicinol in blood of the patient. The carbonyl atom may be modified with a molecule comprising a hydrazide moiety. Preferably, the molecule comprising the hydrazide moiety is N-c-maleimidocaproic acid hydrazide.

In another aspect of the inventive subject matter, the inventors also contemplate a method of treating cancer in a patient that has exceeded a cumulative dose limit of 400 mg/m² of doxorubicin, comprising a step of administering a doxorubicin derivative, wherein the doxorubicin derivative has a reactive group that binds to albumin. In some preferable embodiments, the doxorubicin derivative is aldoxorubicin. The dosage of aldoxorubicin administered is preferably at least 3,000 mg/m², or at least 4,000 mg/m², or at least 5,000 mg/m², or at least 10,000 mg/m². Typically, it is contemplated that the cancer is responsive to administration of doxorubicin. Thus, the cancer may be a solid cancer, such as a small cell lung cancer, an ovarian cancer, a gastric cancer, a bladder cancer, a thyroid cancer, or a breast cancer. The cancer may also be a soft tissue sarcoma, a Kaposi's sarcoma, a glioblastoma, a leukemia, or a lymphoma.

In yet another aspect of the inventive subject matter, the inventors disclose a method of generating doxorubicin cumulative doses of at least 400 mg/m² in a patient, comprising a step of administering a doxorubicin derivative, wherein the doxorubicin derivative has a reactive group that binds to albumin. The albumin may be released from the doxorubicin in an acidic tumor microenvironment, and the doxorubicin cumulative dose is contemplated to not produce cardiotoxicity. The doxorubicin cumulative dose is at least 4,000 mg/m², or at least 7,000 mg/m², or at least 10,000 mg/m².

Also disclosed is a method of reducing alopecia induction in a patient treated with doxorubicin, comprising a step of modifying the doxorubicin to form a carbonyl-modified doxorubicin that binds to albumin, and administering the carbonyl-modified doxorubicin to the patient. The carbonyl-modified doxorubicin may be aldoxorubicin.

Further disclosed herein is a treatment kit, comprising a concentrated solution of aldoxorubicin and a pharmaceutically acceptable diluent suitable for injection. The concentrated solution of aldoxorubicin may comprise a polar aprotic solvent. The concentrated solution of aldoxorubicin is preferably packaged in a prefilled syringe or ampoule for single-use or in a glass or plastic vial for multiple uses. The pharmaceutically acceptable diluent suitable for injection may be a saline solution, a Ringer's solution, or a solution comprising a sugar alcohol. It is further contemplated that the aldoxorubicin in the concentrated solution of aldoxorubicin is present in an amount of at least 50 mg, or at least 100 mg.

In still another aspect of the inventive subject matter, the inventors have disclosed a ready-to-use formulation of aldoxorubicin, wherein the aldoxorubicin is present in a pharmaceutically acceptable diluent suitable for injection, and wherein the aldoxorubicin is present in an amount of at least 50 mg, or at least 100 mg, or at least 250 mg. The formulation may be prefilled in an IV bag.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIGS. 1A and 1B depict the chemical structures of doxorubicin and aldoxorubicin, respectively.

FIG. 2 is a Kaplan-Meier plot for a comparison of aldoxorubicin with doxorubicin.

FIG. 3 is a graph depicting exemplary pharmacokinetic parameters for aldoxorubicin, doxorubicin, and doxorubicinol (doxorubicin metabolite).

### DETAILED DESCRIPTION

The inventors have unexpectedly discovered that aldoxorubicin in the cancer treatment of various patients exhibited no or substantially reduced cardiotoxicity, even where the cumulative dose equivalent to doxorubicin was significantly more than 10× that of the allowable maximum cumulative lifetime dose of doxorubicin, which is 550 mg/m². While not limiting to the inventive subject matter, the inventors contemplate that the lack of apparent cardiotoxicity may be due to the substantially reduced formation of doxorubicinol. Indeed, in at least some samples, there was no detectable doxorubicinol upon administration of aldoxorubicin.

While others have attempted clinical and pharmacokinetic studies of aldoxorubicin with various amounts of success, to the inventors' best knowledge, these studies were limited to maximum cumulative lifetime dosages of less than about 3000 mg/m² of aldoxorubicin. For example Chawla et al evaluated the efficacy and safety of aldoxorubicin vs doxorubicin in patients with advanced soft-tissue sarcoma. See Chawla et al, JAMA Oncol. 2015 December; 1(9):1272-80. Furthermore, Chawla et al also studied the efficacy and safety of aldoxorubicin compared to investigators' choice (IC) of treatment in subjects with soft tissue sarcomas (STS) who have relapsed or were refractory to prior chemotherapy. See Chawla et al, J Clin Oncol. 2017; 35(15_suppl):11000. Unger et al characterized the toxicity profile of (6-maleimidocaproyl) hydrazone derivative of doxorubicin (DOXO-EMCH) to establish a recommended dose for clinical studies and to assess potential anticancer activity. See Unger et al, Clin Cancer Res. 2007 Aug. 15; 13(16):4858-66. Various clinical studies have been conducted as well, such as NCT01514188, NCT02049905, NCT 02014844, NCT02029430, and NCT02235701. In each of these above studies, the maximum total cumulative dosage used was less than about 3000 mg/m².

In this disclosure, the inventors report their surprising discovery that dosages higher than 3000 mg/m², such as dosages of 3,000 mg/m²-4,000 mg/m², or 4,000 mg/m²-5,000 mg/m², or 5,000 mg/m²-6,000 mg/m², or 6,000 mg/m²-7,000 mg/m², or 7,000 mg/m²-8,000 mg/m², or 8,000 mg/m²-9,000 mg/m², or 9,000 mg/m²-10,000 mg/m², or 10,000 mg/m²-15,000 mg/m², and up to 20,000 mg/m² can be administered to patients, with patients exhibiting no or substantially reduced cardiotoxicity, even where these patients may have approached or reached their cumulative lifetime dose of doxorubicin of 550 mg/m².

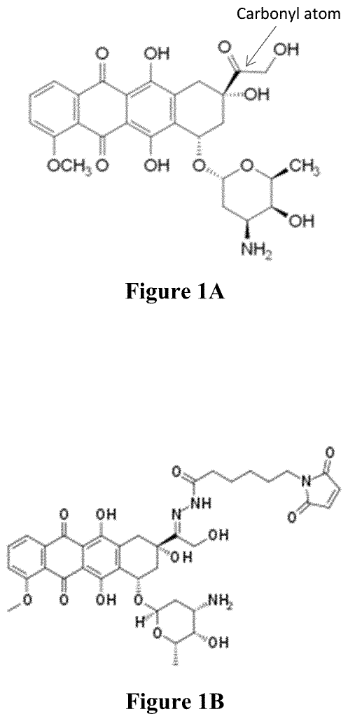

FIG. 1A depicts the chemical structure for doxorubicin, while FIG. 1B depicts the chemical structure for aldoxorubicin. As can be readily seen from these Figures, the modification of the carbonyl atom in the side chain with N-c-maleimidocaproic acid hydrazide provides the linker with a thiol reactive group, but may also provide protection from metabolic conversion of doxorubicin to doxorubicinol. Therefore, the inventors also contemplate various other covalent modifications that are contemplated to reduce such metabolic conversion to doxorubicinol. Among other suitable groups, various amino acids and short or medium chain hydrocarbon residues may be added. Similarly, amino sugars or hydrazide sugars may be suitable for such modification.

Based on the surprising result of lack of cardiotoxicity as shown in further detail below (possibly due to the lack of metabolic conversion of aldoxorubicin after release from albumin), the inventors therefore contemplate cancer (and other) treatments in which aldoxorubicin is administered to a human or other mammal at dosages of 100-200 mg/m², or 200-300 mg/m², or 300-400 mg/m², or 400-500 mg/m², or 500-700 mg/m², or even higher, typically by i.v. injection. Most typically, the injections are spaced apart by at least 1 day, or at least 2 days, or at least 3 days, or at least 5 day, or at least 7 days, or at least two weeks, or at least three weeks, or at least four weeks, or even longer.

Therefore, contemplated suitable cumulative (lifetime) doses without cardiotoxicity are at least 500 mg/m², or at least 750 mg/m², or at least 1,000 mg/m², or at least 1,500 mg/m², or at least 2,000 mg/m², or at least 3,000 mg/m², or at least 5,000 mg/m², or at least 7,500 mg/m², or at least 10,000 mg/m². Viewed form another perspective, the inventors also contemplate that any treatment using aldoxorubicin or other carbonyl-modified doxorubicin may be monitored by observing doxorubicinol and proper dosages of the aldoxorubicin or other carbonyl-modified doxorubicin can be established by maintaining doxorubicinol concentrations at or below predetermined levels indicative of (onset of) cardiotoxicity. As known to a skilled artisan in the art, doxorubicin and its major metabolite doxorubicinol are retained inside cardiac cells, which attributes to its cardiotoxicity. Doxorubicinol may have more profound effect on contraction-relaxation cycle of the cardiac muscles as compared to doxorubicin. For example, doxorubicinol could inhibit RYR2, Na⁺/K⁺ pump on the cell membrane, and proton pump on mitochondria, resulting in the impairment of relaxation. Thus, in one embodiment, side effects of cancer treatments using aldoxorubicin may be monitored by observing doxorubicinol, and appropriate doses of aldoxorubicin may be established through such monitoring process. Due to the modification as disclosed herein, formation of doxorubicinol is expected to occur at significantly reduced rates such as equal or less than 20%, equal or less than 15%, equal or less than 10%, equal or less than 5%, equal or less than 1%, or even lower (all rates compared to formation of doxorubicinol from doxorubicin). This enables administration of higher cumulative amounts of aldoxorubicin as described throughout this disclosure.

Viewed from a different perspective, the inventors also contemplate various compositions and formulations that comprise aldoxorubicin for administration to an individual in need thereof. Most typically, such formulations can be prepared in which aldoxorubicin is provided as a dry composition (e.g., crystalline, lyophilized, freeze dried, etc.) in combination with a diluent. As will be readily appreciated, suitable diluents will be pharmaceutically acceptable solvents and all reasonable mixtures thereof. For example, a suitable solvent for aldoxorubicin may be DMSO or other polar and/or aprotic solvent (e.g., NMP, THF, DMF, ethyl acetate, ethanol), typically in combination with an aqueous base in a ready-to-use formulation, or separately provided. In such case, aldoxorubicin can be provided as a concentrate in an organic non-aqueous solvent for dilution into a saline (or other aqueous) solution prior to administration, or in a ready-to-use formulation in which an aldoxorubicin stock solution is diluted to a concentration suitable for injection.

Most typically, the diluent is therefore at or near physiological pH (e.g., between 4.5-6.0, or between 5.0-6.5, or between 5.5-7.5, or between 6.0-7.5, or between 7.0-8.5, or between 7.5-8.5). Particularly preferred diluents are aqueous diluents with adjusted osmolarity, preferably between 200-1,200 mOsmol/L. Moreover, such diluents may include one or more electrolytes, sugar alcohols, lactate, etc. For example, contemplated diluents include mannitol solutions, lactated Ringer solution, isotonic saline solutions, Ringer's plus dextrose solutions, multi-electrolyte solutions, etc.

With respect to the aldoxorubicin in the stock solution, it is generally preferred that the aldoxorubicin is present in a single dose format and as such present in an amount of 100-200 mg/dose, or 200-300 mg/dose, or 300-400 mg/dose, or 400-500 mg/dose, or 500-700 mg/dose, or 700-1,000 mg/dose, or even higher. Such single dose forms may be provided in a vial or prefilled syringe, which may accompany the diluent as appropriate. On the other hand, the aldoxorubicin may also be provided as a stock solution for multiple uses in an appropriate solvent or mixed solvent system. In such case, quantities of aldoxorubicin in a vial or other container may exceed 500 mg, or 1,000 mg, or 2,000 mg, or 3,000 mg, or 5,000 mg, or 10,000 mg, or even more.

Consequently, aldoxorubicin stock solutions and ready-to-use compositions may be packaged in a single use format (e.g., contains aldoxorubicin sufficient for single administration) or a multi-use format (e.g., contains aldoxorubicin sufficient for at least two administrations), for example, in an ampoule, glass or plastic vial, stoppered bottle, prefilled syringe, IV bag, etc. Likewise, where aldoxorubicin is provided in a stock solution, the stock solution may be accompanied with suitable diluents as discussed above. Typically, in such case the stock solution is in a single use format with a suitable quantity of diluent (e.g., 250 mL, or 500 mL, or 1,000 mL). Regardless of the particular format, it is generally preferred that the aldoxorubicin is provided in a format that allows administration of a single dosage of 100-200 mg/m², or 200-300 mg/m², or 300-400 mg/m², or 400-500 mg/m², or 500-700 mg/m², or 700-1,000 mg/m², or even higher.

In view of the unexpected lack of cardiotoxicity it is contemplated that aldoxorubicin administration can be achieved for patients that had previously received treatment with an albumin binding doxorubicin drug who have exceeded the standard cumulative upper dose limit of 400 mg/m². In these cases, aldoxorubicin can be administered beyond a cumulative dose of 400 mg/m², or cumulative dose of 600 mg/m², or cumulative dose of 800 mg/m², or cumulative dose of 1,000 mg/m², or even higher. Notably, such continued treatment is achievable without cardiotoxicity by continued administration of aldoxorubicin. Therefore, viewed from a different perspective, greater effective dosages can be attained with lower cardio toxicity where doxorubicin is replaced with aldoxorubicin.

Among other indications, it is particularly preferred that aldoxorubicin, and particularly high dosage (cumulative dose of >400 mg/m², or cumulative dose of >800 mg/m², or cumulative dose of >1,500 mg/m², or cumulative dose of >5,000 mg/m², or cumulative dose of >10,000 mg/m²) treatment using aldoxorubicin can be administered to patients diagnosed with various tumors, and especially soft tissue tumors. For example, contemplated tumors include Kaposi's sarcoma, soft tissue sarcoma, glioblastoma, various leukemias, lymphomas, as well as various solid tumors (small cell lung cancer, ovarian cancer, gastric cancer, bladder cancer, thyroid cancer, breast cancer, etc.). In general, all cancers that can be treated with doxorubicin are also deemed suitable for treatment with aldoxorubicin.

Moreover, the inventors also unexpectedly discovered that where aldoxorubicin was administered instead of doxorubicin, patients experienced minimal alopecia induction (typically with hair loss of less than 20%, or less than 15%, or less than 10%, or less than 5%).

### Examples

Fifty-two patients enrolled in a Phase 1/2 study of aldoxorubicin and ifosfamide/mesna and a Phase 3 study using aldoxorubicin alone were treated for at least 6 cycles of aldoxorubicin at either 250 mg/m² or 350 mg/m² per dose i. v. every 3 weeks. Cardiac function using 2D echocardiogram was evaluated at regular intervals every two cycles of aldoxorubicin until end of treatment and every six months after completion of the treatment.

In eleven patients, the median cumulative doxorubicin dose prior to aldoxorubicin treatment was 158 (range: 64-360) mg/m². After treatment, the cumulative aldoxorubicin dose for these patients ranged from 1,000 to 7,500 mg/m². Notably, no patient developed any sign or symptom of clinical congestive heart failure. Ventricular ejection fractions ranged from 45-74% baseline, and 50-77% at end of treatment, median being 60% both at the beginning and end of treatment. Therefore, it should be appreciated that aldoxorubicin lacks cardiotoxicity in these patients treated with aldoxorubicin, alone or in combination with ifosfamide/mesna. Remarkably, the inventors did not find any evidence of cardiac toxicity of aldoxorubicin up to doxorubicin equivalent doses of 7,500 mg/m². As is further shown below, doxorubicin and aldoxorubicin had similar pharmacokinetic parameters with a decline of the drug over time.

Sensitivity Analysis: To assess the impact of censoring the 8 patients with no baseline imaging at the date of randomization, a sensitivity analysis was conducted where a PFS event was imputed at the date of randomization. This analysis is presented in Table 1 showing the sensitivity analysis of PFS (ITT Population^{a}).

TABLE 1

Independent Assessment

| Variable | Doxorubicin | Aldoxorubicin |
|---|---|---|
| Number of Patients | 42 | 84 |
| PFS Events | 29 | 52 |
| Median PFS (Days) | 82.0 | 132.0 |
| (95% CI) | (47.0-121.0) | (81.0-246.0) |

| | |
|---|---|
| P-value^{b} | 0.032 |
| HR (95% CI)^{c} | 0.60 (0.38, 0.95) |

PFS is defined as the time from randomization to the date of first objective documentation of disease progression or death (any cause), whichever comes first. CI=Confidence interval; ECOG=Eastern Cooperative Oncology Group; HR=Hazard ratio; PFS=Progression-free survival; PS=Performance status. ^{a} The ITT Population comprised all randomized patients. ^{b}P-value is from stratified log-rank test with ECOG PS (0 to 1, 2) and chemotherapy (no prior chemotherapy or prior adjuvant/neoadjuvant chemotherapy) as stratification factors. Hazard ratio and 95% CI are from a Cox regression model stratified by ECOG PS (0 1, 2) and chemotherapy (no prior chemotherapy or prior adjuvant/neoadjuvant chemotherapy).

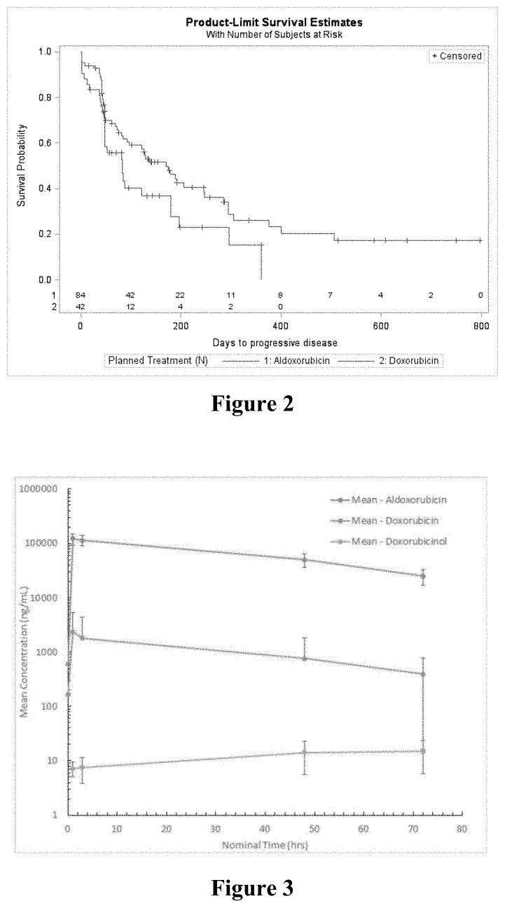

FIG. 2 displays a Kaplan-Meier plot for the sensitivity analysis of PFS. Table 1 indicated that aldoxorubicin provides double the median PFS that was observed for doxorubicin (170 days vs 83 days) and aldoxorubicin was statistically superior to doxorubicin (p=0.032, HR=0.60, 95% CI [0.38, 0.95]).

Worst-Case Sensitivity Analysis: To assess the impact of overall censoring in the primary analysis, a worst-case sensitivity analysis was conducted where a PFS event was imputed at the date of censoring for 17 patients who received doxorubicin and 36 patients who received aldoxorubicin. This analysis is presented in Table 2 (Worst-Case Sensitivity Analysis of PFS (ITT population^{a})).

TABLE 2

Independent Assessment

| Variable | Doxorubicin | Aldoxorubicin |
|---|---|---|
| Number of Patients | 42 | 84 |
| PFS Events | 42 | 84 |
| Median PFS (Days) | 48.0 | 88.5 |
| (95% CI) | (42.0-83.0) | (48.0-129.0) |

| | |
|---|---|
| P-value^{b} | 0.018 |
| HR (95% CI)^{c} | 0.63 (0.43, 0.93) |

PFS is defined as the time from randomization to the date of first objective documentation of disease progression or death (any cause), whichever comes first. CI=Confidence interval; ECOG=Eastern Cooperative Oncology Group; HR=Hazard ratio; PFS=Progression-free survival; PS=Performance status. ^{a}The ITT Population comprised all randomized patients.^{b} P-value is based on a stratified log-rank test with ECOG PS (0 to 1, 2) and chemotherapy (no prior chemotherapy or prior adjuvant/neoadjuvant chemotherapy) as stratification factors. Hazard ratio and 95% CI are from a Cox regression model stratified by ECOG PS (0 1, 2) and chemotherapy (no prior chemotherapy or prior adjuvant/neoadjuvant chemotherapy).

Cardiotoxicity: In a phase 2 study (INNO-206-P2-STS-01), patients treated with aldoxorubicin received more than 5 times the cumulative amount of doxorubicin than the patients treated with doxorubicin.

Remarkably, no clinically relevant decreases in LVEF were observed in the aldoxorubicin treatment group, and in more instances, an increase in LVEF, either by MUGA or echocardiogram, was observed. Similarly, in a phase 3 study (P3-STS-01), aldoxorubicin, given at 350 mg/m²/cycle, was shown to have minimal or no cardiotoxicity up to 40 cycles, as compared to doxorubicin. As shown in Table 3 (LVEF Changes Past Phase 2 and Phase 3 Studies), both studies demonstrated a significantly fewer number of patients with LVEF<50% in the aldoxorubicin treatment group.

TABLE 3

| | | |
|---|---|---|
| Doxorubicin | Aldoxorubicin | p-value^{c} |

Phase 2 (INNO-206-P2-STS-01)^{a}

| Number of subjects | N | 40 | 83 | |
|---|---|---|---|---|
| Subjects with LVEF | n (%) | 3 (8.6) | 0 | 0.0312 |
| < 50% at any post- | | | | |
| baseline cycle^{d} | | | | |
| Subjects with a 10% fall | n (%) | 10 (29.4) | 9 (12.2) | 0.0539 |
| in LVEF^{e} | | | | |

Phase 3 (P3-STS-01)^{b}

| | | | | |
|---|---|---|---|---|
| Number of subjects | N | 47 | 213 | |
| Subjects with LVEF | n (%) | 9 (19.1) | 15 (7) | 0.0213 |
| below 50% of any | | | | |
| post-baseline visit | | | | |
| Subjects with ≥ 20% | n (%) | 5 (10.6) | 9 (4.2) | 0.1428 |
| LVEF decrease from | | | | |
| baseline at any | | | | |
| post-baseline visit | | | | |

^{a}Dox Control Arm received median of 4 cycle of 75 mg/m² (300 mg/m² doxorubicin) Aldox Arm received median of 6 cycles of 350 mg/m² (1500 mg/m² doxorubicin equivalents; ^{b}median cumulative dose of Aldoxorubicin (doxorubicin equivalent 1359.8 mg); ^{c}-value is calculated using Fisher's exact test. ^{d} Percents are based on number of subjects with at least one non-missing LVEF value at any post-baseline cycle. ^{e} Percents are based on number of subjects with at least one change from baseline value at any post-baseline cycle.

Based on the results above, the inventors investigated whether even higher total cumulative doses of aldoxorubicin could be tolerated. To that end, patients were administered total cumulative dosages of up to 3,000 mg/m², up to 4,000 mg/m², up to 5,000 mg/m², up to 6,000 mg/m², up to 7,000 mg/m², up to 8,000 mg/m², up to 9,000 mg/m², up to 10,000 mg/m², up to 11,000 mg/m², up to 12,000 mg/m², up to 13,000 mg/m², up to 14,000 mg/m², and even higher. Remarkably, even doses of 14,000 mg/m² were tolerated without any significant effect on cardiotoxicity and/or LVEF. As described herein, cardiotoxicity may be monitored by monitoring the level of metabolite doxorubicinol in the cardiac cells. Suitable cumulative dosages of aldoxorubicin include those that will not adversely affect LVEF. As such, LVEF of patients receiving aldoxorubicin will typically be in the range of 55% to 70%. Thus, in some embodiments, the patients may have LVEF of 55%-60%, or 60%-65%, or 65%-70%. Less preferably, aldoxorubicin may be administered to the patient until reaching a slightly lower than normal LVEF, say between 50%-54%, or 45%-50%, or 40%-45%.

The methods of treatments disclosed herein are particularly beneficial for patients who have already received their maximum cumulative dose of at least 550 mg/m² doxorubicin. For example, higher dosages of aldoxorubicin treatment as disclosed herein may be particularly useful for a patient who has a second cancer, and the patient was previously treated with a maximum cumulative dose of doxorubicin for his or her first cancer. The methods disclosed herein would also be especially useful for a patient with a recurring cancer, who had already received the maximum cumulative dosage of doxorubicin in the treatment of the first cancer.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

Moreover, all methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of treating cancer in a patient, comprising:
administering a carbonyl-modified doxorubicin to the patient at a cumulative dosage equivalent to doxorubicin of at least 4,000 mg/m²; and
wherein the step of administering the carbonyl-modified doxorubicin does not produce cardiotoxicity.

2. The method of claim 1 wherein the carbonyl-modified doxorubicin is aldoxorubicin.

3. The method of claim 1, further comprising a step of measuring doxorubicinol in blood of the patient.

4. The method of claim 1 wherein the cancer is responsive to administration of doxorubicin.

5. The method of claim 1 wherein the cancer is a solid cancer.

6. The method of claim 5 wherein the cancer is a small cell lung cancer, an ovarian cancer, a gastric cancer, a bladder cancer, a thyroid cancer, or a breast cancer.

7. The method of claim 1 wherein the cancer is a soft tissue sarcoma, a Kaposi's sarcoma, a glioblastoma, a leukemia, or a lymphoma.

8. A method of treating a patient that has exceeded a cumulative dose limit of 400 mg/m² of doxorubicin, comprising a step of administering a doxorubicin derivative, wherein the doxorubicin derivative has a reactive group that binds to albumin.

9. The method of claim 8 wherein the doxorubicin derivative is aldoxorubicin.

10. The method of claim 8 wherein the doxorubicin derivative is administered to a cumulative dose of at least 3,000 mg/m².

11. The method of claim 8 wherein the doxorubicin derivative is administered to a cumulative dose of at least 4,000 mg/m².

12. The method of claim 8 wherein the doxorubicin derivative is administered to a cumulative dose of at least 5,000 mg/m².

13. The method of claim 8 wherein the doxorubicin derivative is administered to a cumulative dose of at least 10,000 mg/m².

14. A method of generating doxorubicin equivalent cumulative doses of at least 4,000 mg/m² in a patient, comprising a step of administering a doxorubicin derivative, wherein the doxorubicin derivative has a reactive group that binds to albumin.

15. The method of claim 14 wherein the albumin is released from the doxorubicin in an acidic tumor microenvironment.

16. The method of claim 14 wherein the doxorubicin cumulative dose is at least 5,000 mg/m².

17. The method of claim 14 wherein the doxorubicin cumulative dose is at least 7,000 mg/m².

18. The method of claim 14 wherein the doxorubicin cumulative dose is at least 10,000 mg/m².

19. The method of claim 14 wherein the doxorubicin cumulative dose does not produce cardiotoxicity.
